(19) United States
(12) Patent Application Publication
(10) Pub. No.: US 2023/0158234 A1
(43) Pub. Date: May 25, 2023

(54) Method of Insulin Administration

(71) Applicant: Colleen Cook, Eden Prairie, MN (US)

(72) Inventor: Colleen Cook, Eden Prairie, MN (US)

(21) Appl. No.: 17/531,836

(22) Filed: Nov. 22, 2021

(51) Int. Cl. A61M 5/172; G16H 20/10

(52) U.S. Cl. CPC A61M 5/1723; G16H 20/10; A61M 2230/20; A61M 2205/52; A61M 2205/50; A61M 2205/502

(57) ABSTRACT

A method of determining initial insulin dosage range for patients with diabetes includes determining insulin dosage range based upon factors of estimated glomerular filtration rate (eGFR) and body mass index (BMI), and administering such dosage to the patient.

### FIELD OF THE DISCLOSURE

The invention relates generally in one embodiment to a method for determining a desired amount of insulin for patients with diabetes; and more specifically, to a method for determining initial insulin dosage range for such patients based upon factors of estimated glomerular filtration rate (eGFR) and body mass index (BMI).

### BACKGROUND

Insulin is an essential hormone produced by the pancreas. Insulin's primary role is to regulate glucose levels in the body. Insulin lowers blood glucose levels by moving glucose from the blood into the cells of the body, where glucose provides energy.

In general, diabetes is a condition that causes the pancreas to make less and less insulin. As a result, diabetes patients are unable to control their blood glucose levels naturally. Diabetes patients thus will take insulin to keep their blood sugar levels in a normal range as much as possible.

Basal insulin, also known as background insulin, mimics the process of the pancreas normally making set amounts of insulin around the clock. The body absorbs basal insulin slowly and uses it throughout the day. People take basal insulin once or twice a day depending on insulin dose to control blood sugar outside of eating.

Prandial insulins, on the other hand, are taken at mealtime and act rapidly in the body, serving to manage the elevation of glucose levels following meals. Prandial insulins can also be used as correction doses (i.e., between meals or during the night) if glucose levels are high and out of range on the high side.

A physician prescribes an insulin dose regimen for patients. Such a regimen typically is determined based upon a patient's body weight.

There remains a need for an improved method of insulin delivery including the step of determining insulin dosage range based on patient factors other than weight.

### SUMMARY

The present disclosure in one embodiment provides a method for determining insulin dosage range for patients with diabetes based upon factors of estimated glomerular filtration rate (eGFR) and body mass index (BMI).

In one embodiment, an individualized insulin dosing tool determines the initial basal or prandial insulin dose range. The healthcare provider will select the dose which can be used as (1) a basal insulin dose with current oral diabetic regimen; or (2) a basal dose and a prandial dose divided equally over 3 meals (e.g., if total prandial insulin dose is 15 units, then 5 units with breakfast, 5 units with lunch and 5 units with dinner). The target audience would be new diabetics with A1C>9 or existing patients with diabetes (on oral diabetic meds, but not on insulin) with A1C>9 or patients new to insulin due to declining renal function. The initial basal insulin dose range option will be used in newly diagnosed diabetics with A1C>9, existing patients with diabetes new to insulin with A1C>9 or patients with eGFR<15. The initial basal and/or prandial insulin doses will be used in patients new to diabetes with A1C>9.

The output from the tool may provide an insulin dosing range which the prescriber can use to select a dose for a patient. For example, if an insulin dose range of 8-12 units is called for, a provider may select a desired amount within that range, e.g., 10 units. The tool is quick, easy, accurate and convenient.

Other benefits and advantages of the present disclosure will be appreciated from the following detailed description.

### DESCRIPTION OF THE DRAWINGS

FIG. 1 is a table showing recommended insulin dose, for non-dialysis patients with an eGFR of 60 or greater, based upon the patient's BMI.

FIG. 2 is a table showing recommended insulin dose, for non-dialysis patients with eGFR less than 60 and greater than 15, based upon the patient's BMI.

FIG. 3 is a table showing recommended insulin dose, for non-dialysis patients with eGFR less than or equal to 15, and for patients on dialysis.

### DETAILED DESCRIPTION

Embodiments of the invention and various alternatives are described. Those skilled in the art will recognize, given the teachings herein, that numerous alternatives and equivalents exist which do not depart from the invention. It is therefore intended that the invention not be limited by the description set forth herein or below.

One or more specific embodiments of the system and method will be described below. These described embodiments are only exemplary of the present disclosure. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, for clarity and convenience only, and without limitation, the disclosure (including the drawings) sets forth exemplary representations of only certain aspects of events and/or circumstances related to this disclosure. Those skilled in the art will recognize, given the teachings herein, additional such aspects, events and/or circumstances related to this disclosure, e.g., additional elements of the tools described; events occurring related to insulin dosing; etc. Such aspects related to this disclosure do not depart from the invention, and it is therefore intended that the invention not be limited by the certain aspects set forth of the events and circumstances related to this disclosure.

In one embodiment of this disclosure, an insulin dosing range is provided based upon at least three factors: (1) whether a patient is on dialysis, (2) estimated glomerular filtration rate (eGFR), and (3) body mass index (BMI).

Dialysis

An individual on dialysis has compromised kidney function. In accordance with this disclosure, a patient who is on dialysis receives a recommended initial insulin dose range of 0.10 to 0.13 insulin units per kilogram of body mass (IU/kg).

Estimated Glomerular Filtration Rate (eGFR)

The kidneys filter blood by removing waste and extra water to make urine. The kidney's filtration rate, called the glomerular filtration rate (GFR), shows how well the kidneys are filtering. However, testing for GFR is complicated. Accordingly, physicians often use a formula to estimate GFR, or eGFR.

A GFR greater than 60 is considered normal for an adult, while an eGFR greater than 90 is considered normal for an adult. Both GFR and eGFR tend to decrease with age.

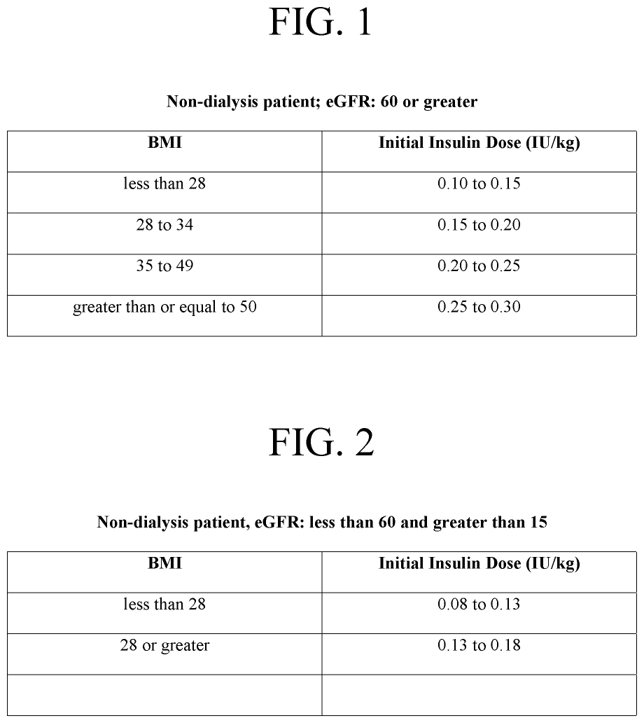

In accordance with this disclosure, a non-dialysis patient with an eGFR of 60 or greater will receive varying initial doses of insulin based upon BMI, as shown in FIG. 1.

In accordance with this disclosure, a non-dialysis patient with an eGFR less than 60 and greater than 15 will receive a lower varying initial dose of insulin based upon BMI, as shown in FIG. 2.

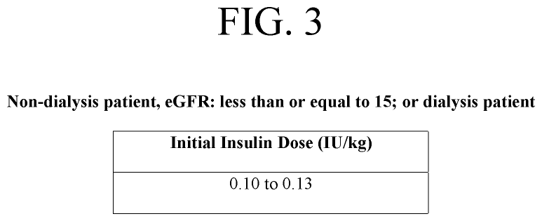

In accordance with this disclosure, both (i) a non-dialysis patient with an eGFR less than or equal to 15, and (ii) a dialysis patient, will receive a lower initial dose of insulin, as shown in FIG. 3.

A method in accordance with this disclosure may include the steps of:

(a) determining a patient's eGFR and BMI;

(b) if the patient is on dialysis, administer to the patient an initial insulin dose of 0.10 to 0.13 IU/kg; or if the patient is not on dialysis, then:

(c) if the non-dialysis patient has an eGFR greater than or equal to 60, then:
(i) if the patient's BMI is less than 28, then administer to the patient an initial insulin dose of 0.10 to 0.15 IU/kg;
(ii) if the patient's BMI is 28 to 34, then administer to the patient an initial insulin dose of 0.15 to 0.20 IU/kg;
(iii) if the patient's BMI is 35 to 49, then administer to the patient an initial insulin dose of 0.20 to 0.25 IU/kg; or
(iv) if the patient's BMI is greater than or equal to 50, then administer to the patient an initial insulin dose of 0.25 to 0.30 IU/kg;

(d) if the non-dialysis patient has an eGFR of less than 60 and greater than 15, then:
(i) if the patient's BMI is less than 28, then administer to the patient an initial insulin dose of 0.08 to 0.13 IU/kg; or
(ii) if the patient's BMI is 28 or greater, then administer to the patient an initial insulin dose of 0.13 to 0.18 IU/kg; and

(e) if the non-dialysis patient has an eGFR of less than or equal to 15, then administer to the patient an initial insulin dose of 0.10 to 0.13 IU/kg.

In one embodiment in accordance with this disclosure, an insulin dosage calculator is provided. The calculator includes a circuit board including circuitry used to execute software code providing instructions for carrying out one or more of the following tasks: (a) inputting patient parameters; (b) calculating one or more eGFR and BMI; and (c) calculating initial insulin dosage range. Patient parameters may include one or more of the following patient data: age, height, weight, sex, A1C (i.e., blood test results that measure one's average blood glucose, or blood sugar, level over the past 3 months), Cr (i.e., blood test results that measure one's level of creatinine in the blood), whether the patient is on dialysis, eGFR, BMI, and race (black or non-black). The calculator includes a memory in which instructions for calculating insulin dosage range are stored. Such instructions will be in accordance with the initial insulin dosing method described herein. The calculator also includes a display for communicating to a user one or more of the patient parameters or calculated eGFR, BMI, or initial insulin dosage range. A method in accordance with this disclosure thus may include the steps of: (a) receiving one or more patient parameters; (b) calculating one or more of eGFR, BMI, and initial insulin dosage range; and (c) displaying one or more of eGFR, BMI, and initial insulin dosage range.

It should be understood that the foregoing description is only illustrative of the invention. Various alternatives and modifications can be devised by those skilled in the art having the benefit of this disclosure, without departing from the invention. Accordingly, the invention is intended to embrace all such alternatives, modifications and variances.

Certain exemplary embodiments of the disclosure may be described. Of course, the embodiments may be modified in form and content, and are not exhaustive, i.e., additional aspects of the disclosure, as well as additional embodiments, will be understood and may be set forth in view of the description herein. Further, while the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention.

What is claimed is:

1. A patient insulin dosing method including the steps of:
(a) determine the patient's estimated glomerular filtration rate (eGFR) and body mass index (BMI);
(b) if the patient is on dialysis, administer to the patient an initial insulin dose of 0.10 IU/kg to 0.13 IU/kg; or if the patient is not on dialysis, then:
(c) if the patient has an eGFR greater than or equal to 60, then:
(i) if the patient's BMI is less than 28, then administer to the patient an initial insulin dose of 0.10 to 0.15 IU/kg;
(ii) if the patient's BMI is 28 to 34, then administer to the patient an initial insulin dose of 0.15 to 0.20 IU/kg;
(iii) if the patient's BMI is 35 to 49, then administer to the patient an initial insulin dose of 0.20 to 0.25 IU/kg; or
(iv) if the patient's BMI is 50 or greater, then administer to the patient an initial insulin dose of 0.25 to 0.30 IU/kg;
(d) if the patient has an eGFR less than 60 to 15, then:
(i) if the patient's BMI is less than 28, then administer to the patient an initial insulin dose of 0.08 to 0.13 IU/kg; or
(ii) if the patient's BMI is 28 or greater, then administer to the patient an initial insulin dose of 0.13 to 0.18 IU/kg; and
(e) if the patient has an eGFR of less than or equal to 15, then administer to the patient an initial insulin dose of 0.10 to 0.13 IU/kg.

2. An initial insulin dosage calculator including:
(a) a circuit board including circuitry executing software code providing instructions for carrying out one or more of the following tasks: (i) inputting patient parameters; (ii) calculating one or more eGFR and BMI; and (iii) calculating initial insulin dosage;
(b) a memory storing instructions for determining initial insulin dosage from one or more patient parameters; and
(c) a display for presenting one or more of initial insulin dosage and patient parameters;
wherein patient parameters include one or more of the following data for a patient: height, weight, A1C, Cr, whether the patient is on dialysis, eGFR, and BMI; and
wherein initial insulin dosage is displayed according to the steps of:
(1) determining the patient's estimated glomerular filtration rate (eGFR) and body mass index (BMI);
(2) determining whether the patient is on dialysis;
(3) if the patient is on dialysis, display an initial insulin dose of 0.10 IU/kg to 0.13 IU/kg;
(4) if the patient is not on dialysis, then:
(x) if the non-dialysis patient has an eGFR greater than or equal to 60, then:
(i) if the patient's BMI is less than 28, then display an initial insulin dose of 0.10 to 0.15 IU/kg;
(ii) if the patient's BMI is 28 to 34, then display an initial insulin dose of 0.15 to 0.20 IU/kg;
(iii) if the patient's BMI is 35 to 49, then display an initial insulin dose of 0.20 to 0.25 IU/kg; or
(iv) if the patient's BMI is greater than or equal to 50, then display an initial insulin dose of 0.25 to 0.30 IU/kg;
(y) if the non-dialysis patient has an eGFR less than 60 and greater than 15, then:
(i) if the patient's BMI is less than 28, then display an initial insulin dose of 0.08 to 0.13 IU/kg; or
(ii) if the patient's BMI is 28 or greater, then display an initial insulin dose of 0.13 to 0.18 IU/kg; and
(z) if the non-dialysis patient has an eGFR of less than or equal to 15, then display an initial insulin dose of 0.10 to 0.13 IU/kg.
